(19) United States
(12) Patent Application Publication
(10) Pub. No.: US 2022/0175841 A1
(43) Pub. Date: Jun. 9, 2022

(54) The Treatment of Protein Aggregation Diseases

(71) Applicant: Pharmakure Limited, Manchester (GB)

(72) Inventor: Christopher Stanley, Liverpool (GB)

(21) Appl. No.: 17/601,542

(22) PCT Filed: Apr. 6, 2020
(86) PCT No.: PCT/GB2020/050900

§ 371 Date: Oct. 5, 2021

(30) Foreign Application Priority Data
Apr. 5, 2019 (GB) 1904810.7

(51) Int. Cl. A61K 35/18; A61P 25/28; C07K 16/18; A61M 1/36

(52) U.S. Cl. CPC A61K 35/18; A61M 1/3679; C07K 16/18; A61P 25/28

(57) ABSTRACT

Compositions, methods and systems for the treatment of a protein aggregation disease including, but not limited to Alzheimer's disease (AD), Parkinson's disease (PD), Dementia with Lewy Bodies (DLB), Huntington's disease (HD), Amylotrophic lateral sclerosis (ALS, which results from degeneration of the upper and lower motor neurones and affects the voluntary muscle system), Progressive Supranuclear Palsy (PSP), Type 2 Diabetes and Multiple systems atrophy (MSA).

More particularly, the present invention relates to compositions, methods and systems for the treatment of a protein aggregation disease including, but not limited to Alzheimer's disease (AD), Parkinson's disease (PD), Dementia with Lewy Bodies (DLB), Huntington's disease (HD), Amylotrophic lateral sclerosis (ALS, which results from degeneration of the upper and lower motor neurones and affects the voluntary muscle system), Progressive Supranuclear Palsy (PSP), Type 2 Diabetes and Multiple systems atrophy (MSA).

In the field of neurodegenerative diseases such as the dementias or Parkinson's Disease there is a particular need to provide treatment. The term dementia covers a large class of neurodegenerative disorders with different causes and overlapping symptoms, of which Alzheimer's disease accounts for 62% of human cases. Each disorder benefits from bespoke management and treatment by clinicians, family and carers, but accurate diagnosis and classification is difficult and is usually based on subjective observations. Degenerative diseases can also include systemic neural forms; an example is amyotrophic lateral sclerosis (ALS) which results from degeneration of the upper and lower motor neurones.

It is known that aggregates of several different types of proteins form in the brain during the progress of a neurodegenerative disease such as Alzheimer's and they may be involved in the pathogenesis of the disease. The detection of plaques or tangles composed of aggregated proteins in the brain by techniques such as imaging in a living subject or by post-mortem histology is considered to be a confirmation of the presence of the specific neurological disease. It is presumed that detection of the early stages of protein aggregation will allow intervention before irreversible neurological damage occurs. The content of brain-derived proteins in cerebrospinal fluid (CSF), such as beta-amyloid (also referred to as Abeta) and tau can aid in the diagnosis and stratification of the relevant dementia diseases. For example the Abeta content of CSF is a promising biomarker in differentiating early AD from normal aging processes as well as allowing prediction of those patients with mild cognitive impairment (MCI), now described as “early Alzheimer's”, who will convert later to moderate to severe Alzheimer's (The Amyloid-Oligomer Count in Cerebrospinal Fluid is a Biomarker for Alzheimer's Disease. Wang-Dietricha, L, Journal of Alzheimer's Disease 34 (2013) 985-994, DOI 10.3233/JAD-122047, which is incorporated herein by reference). A recent paper reported on a 7-year study that showed that the levels of Abeta, total tau and phosphorylated tau in CSF, as measured by standard immunoassays, could be used to predict the onset both of Alzheimer's Disease and its severity (Amyloid imaging and CSF biomarkers in predicting cognitive impairment up to 7.5 years later. Roe et al, Neurology 2013; 80; 1784-1791, which is incorporated herein by reference).

It has been established that the oligomeric forms of certain proteins have neurotoxic properties. These proteins include Abeta (especially the 1-42 form) and alpha synuclein. In Type 2 diabetes the protein amylin (IAPP) also undergoes an oligomerisation process that generates molecular species that are thought to be toxic to islet cells in the pancreas. WO2017067672 discloses that oligomeric and/or aggregated forms of Abeta and alpha synuclein are present in blood and can be detected with a specific analytical procedure. WO2017067672 discloses that blood levels of the oligomers of Abeta and ASN can be elevated in some patients with Alzheimer's Disease, Parkinson's Disease and Dementia with Lewy Bodies. WO2017067672 discloses that oligomeric and/or aggregated forms of proteins are associated with the cellular fraction in blood and plasma levels are much lower.

Therapeutic treatment of such protein aggregation diseases is needed.

According to an aspect of the present invention, there is provided a red blood cell preparation derived from one or more of the following:
i) a donor or donors
ii) the subject's red blood cells or the donor or donor's red blood cells that have been treated ex vivo to reduce the content of protein oligomers and/or aggregates
iii) derived from stem cells or other mononuclear cells
iv) from a xenotransfusion source
   wherein the level of oligomers or aggregates of proteins in the red blood cell preparation has been measured with an analytical method and shown to be at a reduced level and preferably at or below the detection limit of the analytical method.

The present invention is based on the discovery that, in AD and in the Lewy Body disease MSA, a significant proportion of the patient's total body content of the oligomeric forms of certain proteins thought to be involved in protein aggregation diseases is present in the red cell layer after a Ficol gradient separation. Since the vast majority of the cells in this layer are red cells, with very few neutrophils and granulocytes present, it can be assumed that the oligomeric forms are red-cell associated. Using an analytical procedure that uses whole, unfractionated blood elevated levels of the presumed toxic oligomeric form of Abeta have been detected in older (>50 years), outwardly healthy subjects and in Alzheimer's patients and in patients suffering from the Lewy Body diseases Parkinson's and MSA. Similarly, elevated levels of the presumed toxic oligomeric form of alpha-synuclein have been detected with the same analytical procedure in the blood of Alzheimer's, Parkinson's and MSA Disease patients. Since the surface area of the red cells is so high it is proposed that these cells form a major reservoir of the toxic oligomeric proteins in the older, outwardly healthy subjects and in the AD, PD and MSA patient's body in these diseases. The objective of the therapeutic apheresis step of the invention is to deplete the patient's body of the presumed toxic reservoir of these oligomeric and/or aggregated proteins. These protein aggregation diseases develop slowly in AD, PD, MSA and also DLB and other related diseases over many years and so younger donors are preferred as they have been shown by the analytical procedure used in the invention to have much lower blood levels of the presumed toxic oligomeric and aggregated forms.

WO 2011070174 reports the presence of various forms of the Abeta peptide in blood which may be a reliable indicator of the initial development and progression of Alzheimer's disease. It is stated that a proportion of the Abeta peptides in blood are associated with blood cells but the presence of the oligomeric or aggregated forms on red cells is not described. US 20110263450 describes the use of surface enhanced laser desorption time of flight (SELDI-TOF) mass spectrometry to show that higher concentrations of Abeta dimers are present in the blood of some Alzheimer's patients and the dimers are associated with the membranes of blood cells. In US 20110263450 there is no mention of oligomers larger than dimers being present in blood and there is no distinction reported between the red blood cells and other blood cells, additionally it is stated that oligomeric toxic forms of proteins bind to lipid membranes.

There is increasing interest in the use of blood plasma therapy in the treatment of diseases of ageing, including the neurodegenerative diseases. The hypothesis is that plasma from young subjects contains factors that are able to stimulate neurogenesis in older subjects. In a mouse model an heterochronic blood exchange study has shown that blood from an older mouse has a rapid inhibitory effect on neurogenesis and stimulates inflammation in tissues in the linked younger mouse; indicating perhaps that the older blood composition is deleterious in some way to the younger animal (Rebo et al. A single heterochronic blood exchange reveals rapid inhibition of multiple tissues by old blood DOI:10.1038/ncomms13363). Rebo et al speculate that it would be interesting to separate the humeral and cellular influences in their heterochronic blood exchange model, but they do not provide any information on the properties of the cellular fraction. WO2018200560 describes a dosage regimen for treating a patient with cognitive impairment that involves regular treatment with a plasma fraction or plasma protein fraction, preferably derived from a young donor source and preferably on subsequent days over a 5 to 7 day period (on a pulsed dosing basis). US 2018110839 also describes treating patients diagnosed with a cognitive disorder using various plasma fractions including albumin from younger donors. Neither WO201800560 nor US 2018110839 disclose the use of cell fractions from blood and in particular the use of red cells in the treatment of older subjects is not described. U.S. Pat. No. 7,935,252 describes removing the patient's plasma and treating it in an ex vivo process using binding receptors such as antibodies to specifically remove Amyloid precursor protein (APP) and Abeta 1-40 and Abeta 1-42. The depleted plasma is injected back into the patient.

An aspect of the present invention involves removal and replacement of some or all of the subject's red blood cells and replacing them with the red cell preparation using a therapeutic apheresis procedure where the replacement red cells in the preparation are derived from different sources including donors preferably under the age of 50 years, more preferably under the age of 40 years or more preferably under the age of 30 years; or derived from stem cells or from other mononuclear cells; or from a xenotransfusion source such as the pig or other mammals that possess erythrocytes with similar characteristics to human; or from the subject in an autologous process.

In the case of an autologous therapeutic apheresis procedure, red blood cells are removed from the subject and subjected to an ex vivo process to remove the oligomeric or aggregated forms of certain proteins, and then the treated red cell preparation is administered to the subject. The ex vivo process can involve the use of compounds that reverse the aggregation status of oligomeric and aggregated proteins, in this case the compounds and the resulting monomeric forms can be removed in a dialysis or other washing procedure, such as centrifugation, prior to administering the treated red blood cell preparation to the subject. Optionally the monomeric forms of the proteins that result from this treatment can be removed by an affinity adsorbtion step using a specific binding agent such as an antibody or a synthetic antibody such as an aptamer or a specific binding polymer prior to administering the further treated blood cell preparation to the subject. The monomers can also be separated from red cells using non-specific binding techniques such as ion-exchange or size exclusion chromatography.

Compounds suitable for reversing the aggregation status of oligomeric and aggregated proteins include polymers such as pentosan polysulphate, which has been shown to have effects on prion protein aggregation and has been used in the treatment of prion disease. Similar polyionic compounds such as heparan sulphate are known to bind to aggregated proteins such as Abeta and alpha synuclein and may therefore be able to affect the interaction of oligomers with red cell membranes. Other polyionic polymers such as heparin are known to inhibit protein aggregation. Small molecules such as the phenothiazines and the bicyclic and the tricyclic pyridones disclosed in U.S. Pat. No. 6,030,984 are known to affect the aggregation status of proteins including Abeta. Other compounds such as chlorpromazine and quinacrine have been shown to affect aggregation status of prion protein and have been proposed for the treatment of prion diseases, they are thought to affect protein aggregation status. The compounds described in U.S. Pat. No. 8,383,617 have been shown to convert oligomeric forms of Abeta to the monomeric forms. In U.S. Pat. No. 8,383,617 it is shown that certain compounds can reduce aggregates in vitro and can reduce the content of plaques in mouse brain. The optimal compound described in U.S. Pat. No. 8,383,617 for aggregation reversal is the dibenzodiazipene trimipramine which has a tricyclic structure.

The ex vivo process can also be used with blood from donors that have higher levels of oligomers or aggregates present initially that would otherwise not be suitable for the therapeutic apheresis procedure.

Optionally the ex vivo process involves the removal of leukocytes prior to the addition of disaggregation compounds. This can be achieved by size filtration, continuous or intermittent flow centrifugation or the use of specific leukoreduction filters. This step also serves to remove any toxic oligomeric forms of proteins that may be associated with the leukocyte membranes.

The analytical method determines the presence and level of the oligomeric forms of certain proteins in the blood of the patient prior to commencing treatment and monitors the reduction in the level as the red cell replacement therapy progresses. The analytical method can be used to determine when the red cell replacement therapy should be repeated if levels of the oligomers exceed a predefined threshold. The analytical method can also be used to confirm that the source of the red cells is free of the oligomeric forms and can also be used to confirm that the ex vivo treatment process of donor or subject blood has been successful in reducing the levels of the oligomeric or aggregated protein forms present.

In the neurodegenerative diseases it is established that the oligomeric forms of certain proteins have neurotoxic properties. These proteins include Abeta (especially the 1-42 form) and alpha synuclein. In Type 2 diabetes the protein amylin (IAPP) also undergoes an oligomerisation process that generates molecular species that are thought to be toxic to islet cells in the pancreas. WO2017067672 discloses that oligomeric and/or aggregated forms of these proteins are present in blood and can be detected with a specific analytical procedure. WO2017067672 discloses that blood levels of the oligomers of Abeta and ASN can be elevated in some patients with Alzheimer's Disease, Parkinson's Disease and Dementia with Lewy Bodies. WO2017067672 discloses that oligomeric and/or aggregated forms of proteins are associated with the cellular fraction in blood and plasma levels are much lower, but does not provide direct evidence of any association with the red cells fraction.

The Applicant has discovered that the majority of the oligomeric forms of certain proteins thought to be involved in certain protein aggregation diseases are red blood-cell associated. The presumed toxic oligomeric form of Abeta has been detected in the red cell fraction of older (>50 years), outwardly healthy subjects and in Alzheimer's and MSA Disease patients. Similarly the presumed toxic oligomeric form of alpha-synuclein has been detected in the red cell fraction of Alzheimer's and MSA Disease patients. The presumed toxic oligomeric form of Abeta and alpha-synuclein has also been detected in unfractionated blood of Parkinson's Disease patients and it is inferred that this form is also red-cell associated. Since the surface area of the red cells is so high it is proposed that these cells form the major reservoir of the toxic oligomeric proteins in the patient's body in these diseases. The therapeutic apheresis step of the present invention depletes and/or reduces the older patient's body of the presumed toxic reservoir of these oligomeric and/or aggregated proteins. These protein aggregation diseases develop slowly in AD, PD, DLB and other related diseases over many years and so younger donors are preferred as they have been shown by the analytical procedure used in the invention to have much lower blood levels of the presumed toxic oligomeric and aggregated forms.

The donor or donors are preferably under the age of 50 years, more preferably under the age of 40 years or more preferably under the age of 30 years to ensure a lower content of oligomers or aggregates.

According to another aspect of the present invention, there is provided a red blood cell preparation for administration to a subject in a therapeutic apheresis procedure derived from one or more of the following:
i) a donor or donors
ii) the subject's red blood cells or the donor or donor's red blood cells that have been treated ex vivo to reduce the content of protein oligomers and/or aggregates
iii) derived from stem cells or other mononuclear cells
iv) from a xenotransfusion source
   wherein the level of protein oligomers or aggregates in the administered red blood cells has been measured with an analytical method and shown to be at a reduced level and preferably at or below the detection limit of the analytical method.

According to a further aspect of the present invention, there is provided a method of treating a protein aggregation disease in a subject comprising administering red blood cells derived from one or more of the following:
i) a donor or donors
ii) the subject's red blood cells or the donor or donor's red blood cells that have been treated ex vivo to reduce the content of protein oligomers and/or aggregates
iii) derived from stem cells or other mononuclear cells
iv) from a xenotransfusion source
   wherein the level of protein oligomers or aggregates in the administered red blood cells has been measured with an analytical method and shown to be at a reduced level and preferably at or below the detection limit of the analytical method.

According to a further aspect of the present invention, there is provided a method of treating a protein aggregation disease comprising administering red blood cells to the subject derived from one or more of the following sources:
i. a donor or donors under the age of 50 years
ii. the subject's red blood cells that have been treated ex vivo to remove toxic oligomers and/or aggregates
iii. derived from stem cells or other mononuclear cells
iv. from a xenotransfusion
   wherein an assay is used to confirm that the level of toxic oligomers or aggregates in the red blood cells to be administered is at or below the detection limit of the analytical method and the analytical method is used subsequently to detect an increase in the levels in the blood of the subject above a pre-defined threshold thereby triggering a repeat of the therapeutic apheresis process.

The oligomeric or aggregated proteins measured can include, but are not limited to, Abeta, alpha synuclein, DJ-1 (also known as Park7, a protein thought to be associated with Parkinson's Disease), tau, superoxide dismutase (SOD, a protein associated with ALS) or IAPP.

Oligomeric forms of proteins may range in size from dimers up to structures containing 10 to 20 units, multimeric associations of protein subunits may contain from 20 to 100 units, whilst aggregates can be defined as larger structures that can include hundreds, or even thousands of protein molecules. The proteins that form the oligomers and aggregates may be in a native, normally folded form or they can be misfolded or abnormal in structure and therefore may be more prone to aggregation.

In accordance with another aspect of the present invention, there is provided a method of preventing, ameliorating or treating a protein aggregation disease comprising the removal of aggregated proteins from the surface of red blood cells.

The removal of the aggregated proteins from the surface of the red blood cells advantageously takes place ex vivo.

In accordance with another aspect, there is provided a method for removing aggregated proteins from the surface of a red blood cell comprising contacting a subject's red blood cells with means to remove protein aggregates from the surface of a cell.

The means to remove the protein aggregates from the surface of a red blood cell may comprise an antibody or antibody fragment or synthetic antibody capable of binding to the aggregated protein of interest.

The antibody, antibody fragment or synthetic antibody may be immobilised on a substrate. The substrate may comprise any one or more of the following: membrane filters, magnetic or non-magnetic beads of diameter preferably in the range 0.1 μm to 150 μm or monolithic high surface area substrates.

The method may further comprise separating the red blood cell from the aggregated protein.

The red blood cell may then be reintroduced into a subject.

In accordance with a further aspect of the present invention, there is provided a system for use in the treatment of an aggregated protein disease comprising means to bind to aggregated protein of interest which itself is associated with a red blood cell, said means immobilised on a substrate.

The system as described hereinabove may further comprise the step of separating aggregated protein from the red blood cell.

In accordance with a further aspect of the present invention, there is provided a method of removing aggregated proteins from the surface of red blood cells for use in the treatment of an aggregated protein related disease.

In accordance with another aspect of the present invention, there is provided an in vitro method of removing aggregated proteins from the surface of red blood cells, the method comprising the steps of:

i) contacting a blood sample with an immobilised antibody or antibody fragment
ii) washing to remove unbound material to leave immobilised red blood cells bound
iii) releasing the red blood cells from the immobilised antibody or antibody fragment

In accordance with an aspect of the present invention, there is provided a red blood cell preparation obtainable from a subject's red blood cells having been treated with a protein oligomer-disaggregating compound and wherein said compound has been removed.

The present invention will now be described, by way of example only, with reference to the following Examples and Figures.

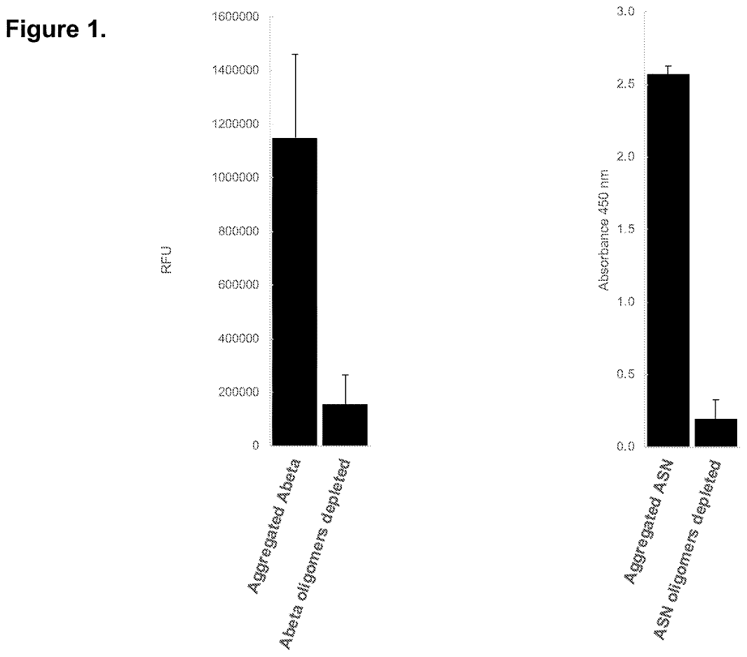

FIG. 1 shows oligomer depletion with A11 antibody prior to using the full assay protocol.

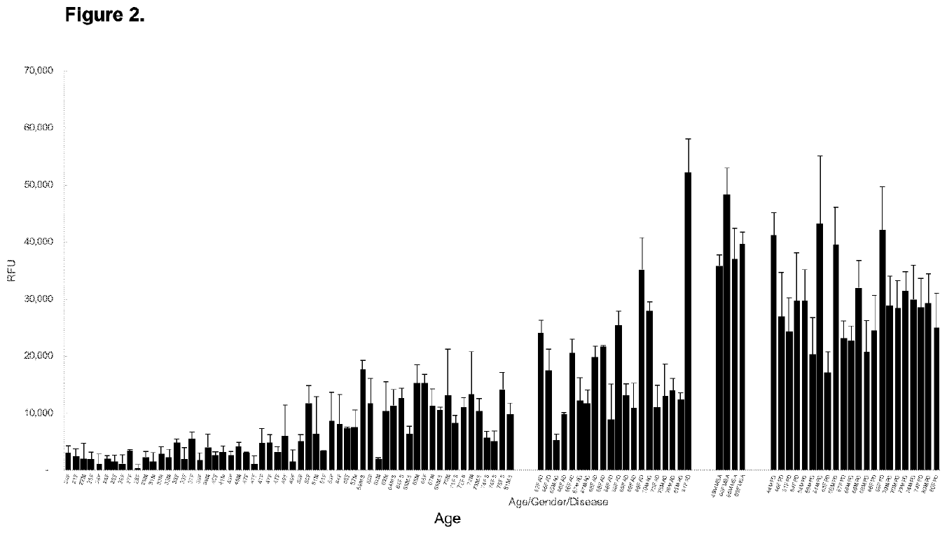

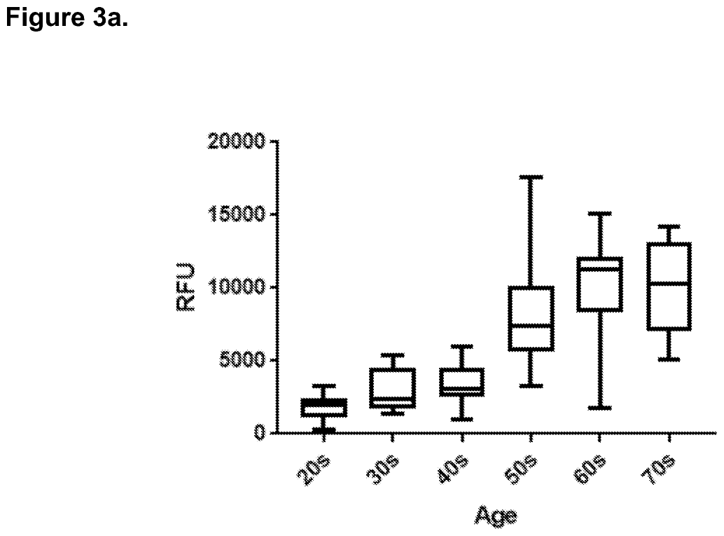

FIG. 2 shows the levels of oligomeric Abeta in whole blood samples from healthy normal controls and from Alzheimer's Disease (AD), Multiple Systems Atrophy (MSA) and Parkinson's Disease (PD) patients. The X axis shows age and gender of the controls and patients, CS' indicates a sample from an age matched spouse. Y axis shows the signal obtained from the oligomeric Abeta assay protocol using a Europium label as relative fluorescence units (RFU) as measured by an LFB-Wallac time-resolved fluorimeter; for the oligomeric ASN study the Y axis shows the signal obtained using an HRP enzyme conjugate, in this case the signal is reported as optical density as measured in a Biotek Inc. colorimetric microplate reader.

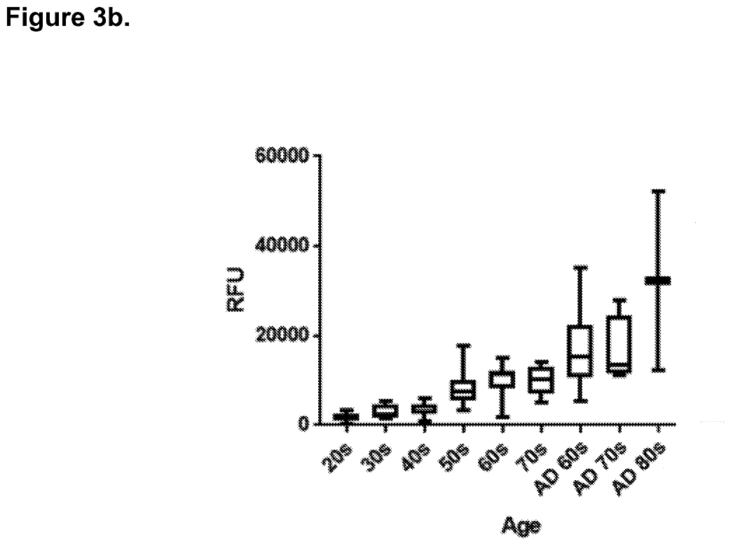

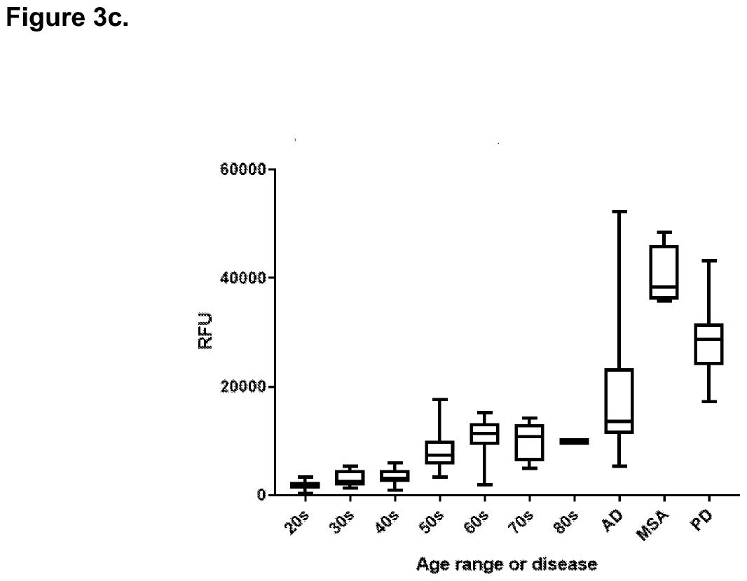

FIG. 3a shows an analysis of the control samples as a box and whisker plot and confirms that there is a rise in the median level of oligomeric Abeta with advancing age.

FIG. 3b shows a box and whisker plot with the added data from the AD patients with advancing age and also includes the MSA and PD patients.

FIG. 3c shows a box and whisker plot with the control samples as well as the AD, MSA and PD patients.

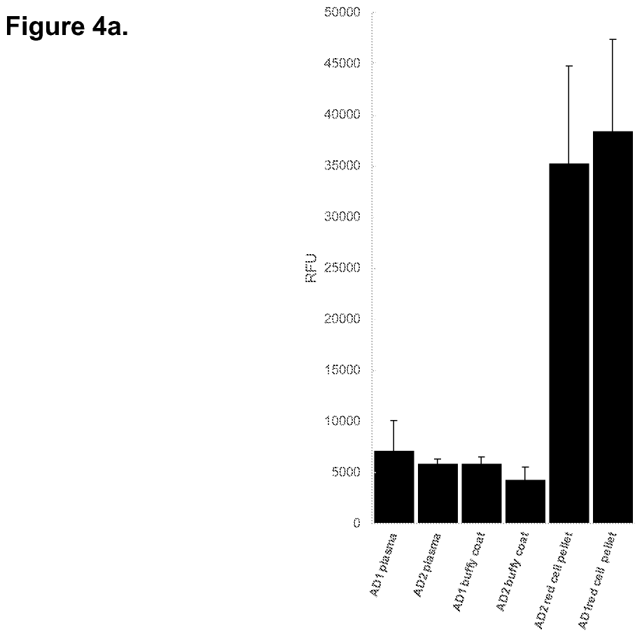

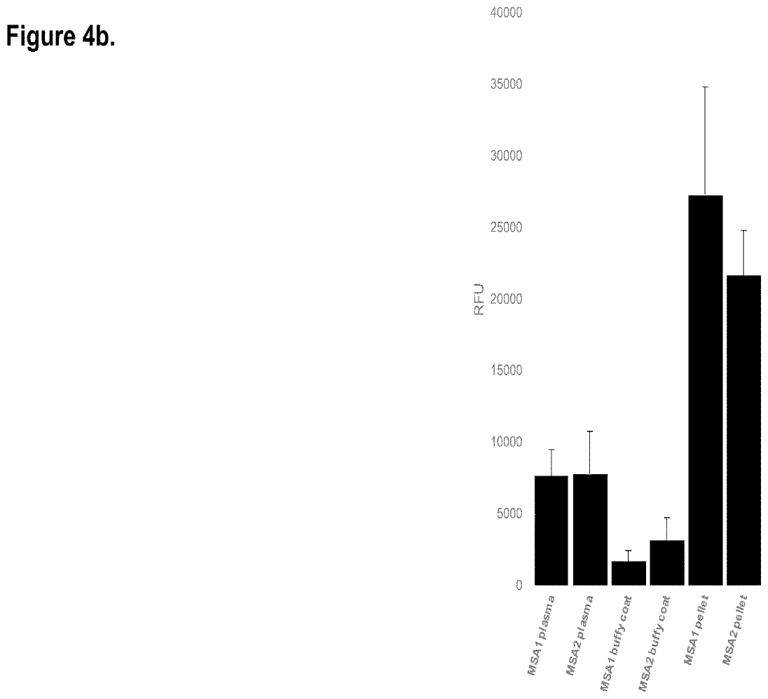

FIG. 4a shows the results from a Ficol gradient separation of whole blood from two AD patients and shows the levels of oligomeric Abeta in the three fractions; plasma, buffy coat (leukocytes) and red cells.

FIG. 4b shows the results from a Ficol gradient separation of whole blood from two MSA patients and shows the levels of oligomeric Abeta in the three layers; plasma, buffy coat (lymphocytes) and predominantly red cells.

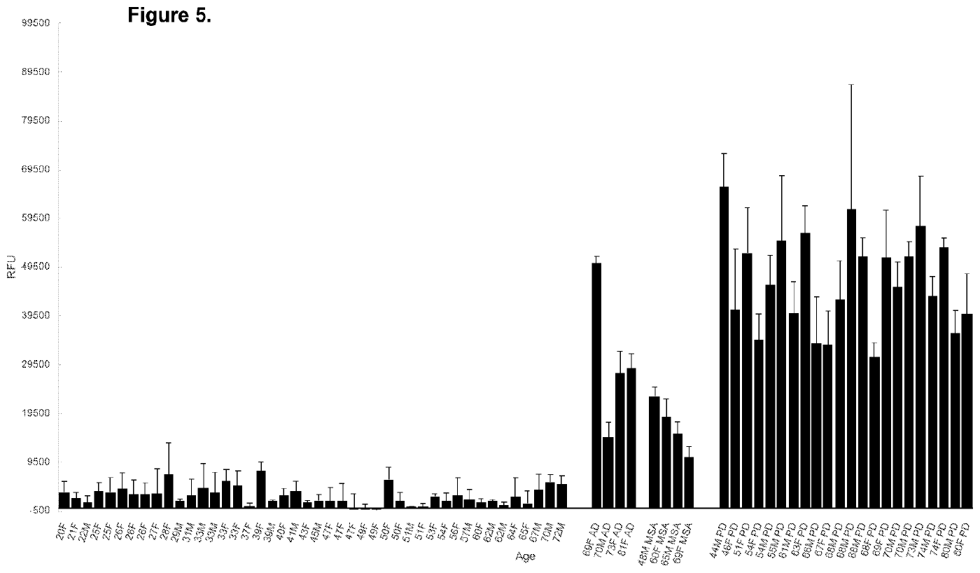

FIG. 5 shows the levels of oligomeric ASN in whole blood samples from healthy normal controls and from AD, MSA and PD patients. The X axis shows age and gender of the controls and patients.

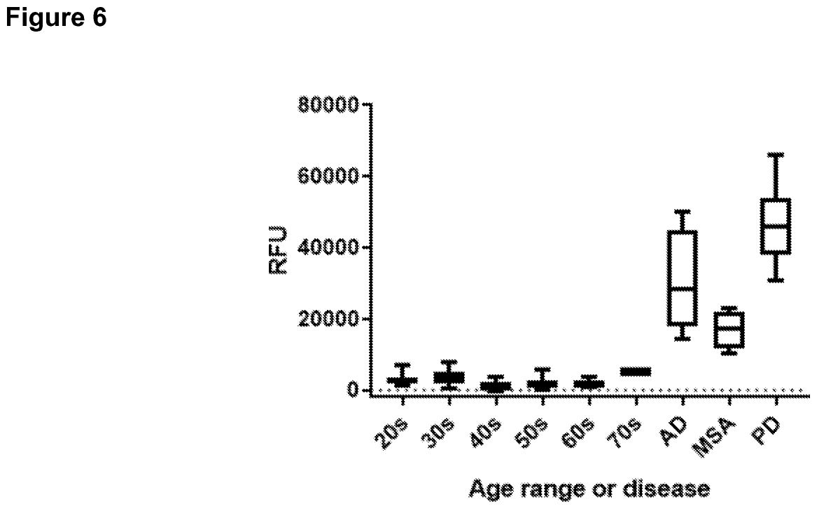

FIG. 6 shows an analysis of the control samples and AD and MSA as a box and whisker plot and confirms that there is no observable rise in the median level of oligomeric ASN with advancing age whilst AD, MSA and PD patients exhibited increases in oligomeric ASN.

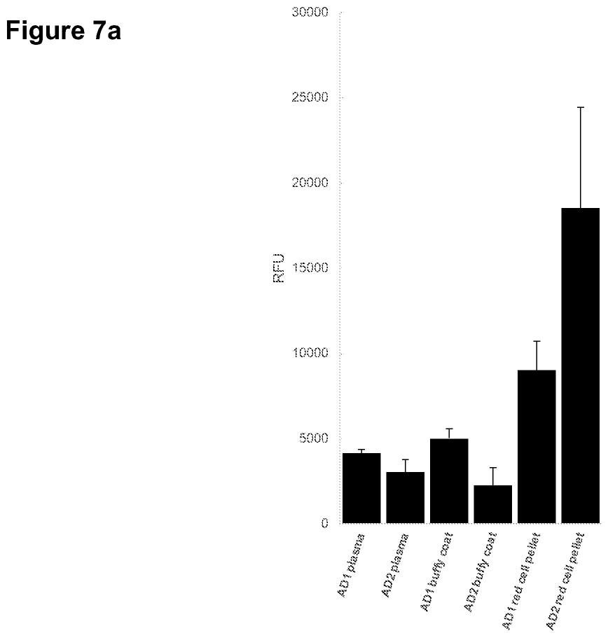

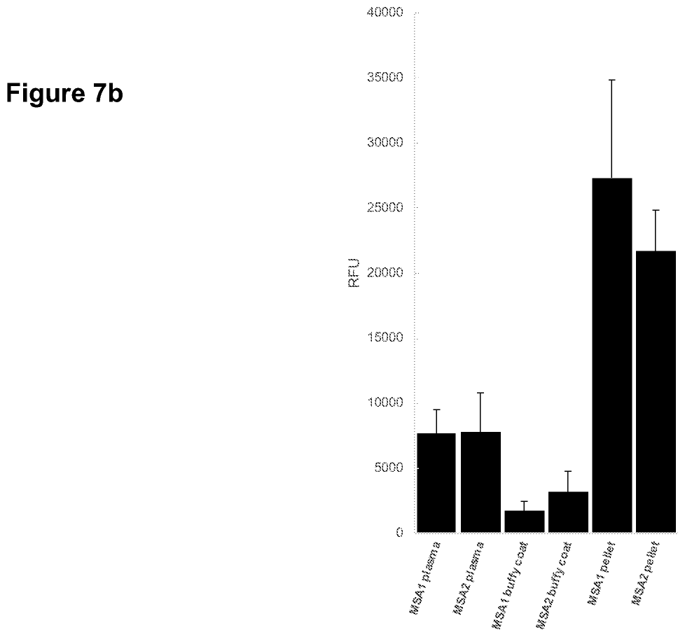

FIG. 7a shows the results from a Ficol gradient separation of whole blood from two AD patients and shows the levels of oligomeric ASN in the three layers; plasma, buffy coat (lymphocytes) and predominantly red cells.

FIG. 7b shows the results from a Ficol gradient separation on whole blood from two MSA patients and shows the levels of oligomeric ASN in the three layers; plasma, buffy coat (lymphocytes) and predominantly red cells.

### EXAMPLES

### Example 1. Oligomer Depletion with all Antibody

Abeta 1-42 and ASN were aggregated according to the methods described in WO2017067672. Protein A-coated magnetic beads (GE Healthcare Inc.), 37-100 μM in diameter, were derivatised with A11 antibody from Thermo Fisher Scientific Inc (2 μl of A11 antibody added to 50 μl of bead suspension) and then added to 1 ml of 100 μg/ml aggregated protein solution in PBS buffer pH 7.5. The mixture was then placed on a rotating incubator for 1 h at room temperature. The tubes were transferred to a magnetic separator and the depleted supernatant was collected for further processing in the full assay protocol described in WO2017067672. A11 has specificity for the oligomeric forms of both Abeta and ASN but does not distinguish between these proteins, most likely the antibody recognizes the conformation of the peptide backbone in an oligomer. FIG. 1 shows that the signal in the assay was reduced to background after treatment with A11-coated beads thus indicating that the full assay protocol was measuring the oligomeric forms of these proteins only.

### Example 2

The full assay protocol described in WO2017067672 was applied to frozen whole blood samples collected from healthy normal controls and from Alzheimer's Disease (AD), Multiple Systems Atrophy (MSA) and Parkinson's Disease (PD) patients. Controls were obtained from healthy volunteers and were collected with full ethical permission by the Liverpool BioInnovation Biobank, UK. Control blood samples were also supplied by Tissue Solutions, Glasgow, UK. Further control samples from age matched spouses were supplied by Salford Royal Infirmary, UK. Results from the Abeta assay protocol are shown in FIG. 2 and the ASN assay protocol are shown in FIG. 5.

### Example 3

A Ficoll (GE Healthcare) gradient separation of fresh whole blood (not frozen) from AD and MSA patients was carried out using the manufacturer's instructions. An aliquot of 1 ml volume was taken from each layer within the gradient and analysed in the full oligomeric protein assay protocol. FIGS. 4a and 4b show the results from the full assay protocol on each layer from the gradient.

1. A red blood cell preparation derived from one or more of the following:
i) a donor or donors;
ii) the subject's red blood cells or the donor or donor's red blood cells that have been treated ex vivo to reduce the content of protein oligomers and/or aggregates;
iii) stem cells or other mononuclear cells; and
iv) from a xenotransfusion source,
wherein the level of oligomers or aggregates of proteins in the red blood cell preparation has been measured and shown to be at a reduced level.

2. (canceled)

3. A red blood cell preparation as claimed in claim 1 wherein the level of protein oligomers or aggregates in the administered red blood cells has been measured with an analytical method and shown to be at a reduced level below the detection limit of the analytical method.

4. A method of treating a protein aggregation disease in a subject comprising administering red blood cells derived from one or more of the following:
i) a donor or donors
ii) the subject's red blood cells or the donor or donor's red blood cells that have been treated ex vivo to reduce the content of protein oligomers and/or aggregates
iii) derived from stem cells or other mononuclear cells
iv) from a xenotransfusion source
wherein the level of protein oligomers or aggregates in the administered red blood cells has been measured and shown to be at a reduced level.

5. The method according to claim 4, wherein the red blood cells are derived from one or more of
i) a donor or donors under the age of 50 years;
ii) the subject's red blood cells that have been treated ex vivo to remove toxic oligomers and/or aggregates;
iii) derived from stem cells or other mononuclear cells; and
iv) from a xenotransfusion,
wherein an assay is used to confirm that the level of toxic oligomers or aggregates in the red blood cells to be administered is at or below the detection limit of the analytical method and the analytical method is used subsequently to detect an increase in the levels in the blood of the subject above a pre-defined threshold thereby triggering a repeat of the therapeutic apheresis process.

6. A method as claimed in claim 4 wherein the level of protein oligomers or aggregates in the administered red blood cells has been measured with an analytical method and shown to be at a reduced level below the detection limit of the analytical method.

7. A red blood cell preparation according to claim 1, wherein the oligomeric or aggregated proteins comprise any one or more of the following: Abeta, alpha synuclein, DJ-1 (also known as Park7), tau, superoxide dismutase (SOD), or IAPP.

8. A method of treating a protein aggregation disease comprising removing aggregated proteins from the surface of red blood cells.

9. The method as claimed in claim 8, wherein the removal of the aggregated proteins from the surface of the red blood cells takes place ex vivo.

10. A method for removing aggregated proteins from the surface of a red blood cell comprising contacting a subject's red blood cells with means to remove protein aggregates from the surface of a cell.

11. The method as claimed in claim 10, wherein the means to remove the protein aggregates from the surface of a red blood cell comprises an antibody, an antibody fragment and/or synthetic antibody capable of binding to the aggregated protein of interest.

12. The method as claimed in claim 11, wherein the antibody, antibody fragment and/or synthetic antibody is immobilised immobilized on a substrate.

13. The method as claimed in claim 12, wherein the substrate comprises any one or more of the following: membrane filters, magnetic beads, non-magnetic beads or monolithic high surface area substrates.

14. The method as claimed in claim 13, wherein the substrate comprises magnetic and/or non-magnetic beads having a diameter in the range 0.1 μm to 150 μm.

15. The method as claimed in claim 14, further comprising separating the red blood cell from the aggregated protein.

16. The method as claimed in claim 15, comprising the subsequent step of reintroducing the red blood cells into a subject.

17-20. (canceled)
